(19) United States
(12) Patent Application Publication
(10) Pub. No.: US 2026/0124058 A1
(43) Pub. Date: May 7, 2026

(54) Height-adjustable back correction belt

(71) Applicants: FIT GENO LLC, NEW YORK, NY (US); SHENZHEN GLOBAL HUITONG SUPPLY CHAIN MANAGEMENT CO., LTD, Shenzhen (CN)

(72) Inventors: Hyewon Ham, NEW YORK, NY (US); Chunmei Liu, Shenzhen (CN)

(21) Appl. No.: 19/438,291

(22) Filed: Dec. 31, 2025

(51) Int. Cl. A61F 5/02

(52) U.S. Cl. CPC A61F 5/028

(57) ABSTRACT

A height-adjustable back correction belt includes a waistband, shoulder straps are disposed on two sides of the waistband, and a strap sleeve is mounted between the two shoulder straps in a penetrating manner. A fixed portion is disposed in a middle of an upper portion of the waistband, a sliding portion is disposed on a rear side of the strap sleeve, the sliding portion is located above the fixed portion, a fixing member is extended through and cooperatively engaged with the fixed portion and a sliding groove of the sliding portion to form a lifting fit structure, and a limiting assembly is disposed between the fixing member and the sliding groove. A waist compression strap is disposed on a middle vertical support strap, and the waist compression strap is pulled to shorten and tighten the shoulder straps backward. The limiting assembly achieves precise locking after height adjustment.

### TECHNICAL FIELD

The present disclosure belongs to the technical field of human posture correction appliances, and particularly relates to a height-adjustable back correction belt.

### BACKGROUND

As an important product in the field of human posture correction appliances, back correction belts are widely used in daily office work, study, rehabilitation training, and other scenarios. The back correction belts mainly function to correct poor sitting and standing postures through applying external forces, which reduce spinal pressure, prevent and alleviate symptoms such as hunchback and scoliosis, and are of great significance for maintaining skeletal and muscular health.

However, back correction belts in the prior art still have the following defects in practical application:

1. Back supports of back correction belts in the prior art are mostly of an integrated fixed-length design, and differences in heights and body shapes of different users are not fully considered. For example, Fixed-length supports cannot flexibly adapt to different users, because there exist significant differences in back lengths and spinal physiological curves between adults and adolescents, and between tall and petite individuals. For users with longer backs, supports are limited in coverage, and hardly effectively support key correction areas such as thoracic and lumbar vertebrae. For users with shorter backs, overlong supports easily lead to redundant restraints, compress body parts such as shoulder blades and waists, and cause soreness and discomfort, and even disuse due to poor wearing experience, thereby directly compromising the correction effect.

2. Although some back correction belts are provided with adjustment mechanisms as an attempt, most of them employ simple means of elastic band stretching or hook-and-loop fastener pasting for adjustments, which only achieves the adjustment of overall tightness, and has deficiencies of failure to precisely control height positioning of back supports, and easy displacement and loosening after the adjustment. Moreover, complicated adjustments and repeated disassembly and adjustment result in poor user convenience.

3. Support structures of back correction belts in the prior art mostly employ an integrated design of rigid materials, thereby resulting in lack of flexible adjustment space, easy localized pressure concentration for different back heights, and compression damage to the skin of shoulders and waists during long-term wearing. Additionally, poor compatibility between rigid support and dynamic human movement affects the flexibility of daily activities.

Therefore, there is an urgent need for a back correction belt capable of flexibly adjusting a back support height to enhance adaptability and user experience.

### SUMMARY

In order to overcome the above deficiencies in the prior art, the present disclosure provides a technical solution to solve the above problems.

A height-adjustable back correction belt includes a waistband, where shoulder straps are disposed on two sides of an upper portion of the waistband, and a strap sleeve is mounted between the two shoulder straps in a penetrating manner; and

a fixed portion is disposed in a middle of an upper portion of the waistband, a sliding portion is disposed on a rear side of the strap sleeve, the sliding portion is located above the fixed portion, a fixing member is disposed in the fixed portion, a sliding groove is formed in the sliding portion, the fixing member is installed in the sliding groove in a liftably and slidably fitted manner, and a limiting assembly is disposed between the fixing member and the sliding groove.

Further, the waistband is made of a fabric with a hook-and-loop fastener loop patch, a back side of either of the shoulder straps is provided with a first hook-and-loop fastener hook patch, an end of a back side of the waistband is provided with a second hook-and-loop fastener hook patch, and both the first hook-and-loop fastener hook patch and the second hook-and-loop fastener hook patch are detachably fixed on the hook-and-loop fastener loop patch of the waistband.

Further, a plurality of vertical support straps are arranged between the waistband and the fixed portion, and support strips are embedded in the vertical support straps.

Optionally, the support strips are made of a metallic or non-metallic material, and the metallic material is any one of stainless steel, aluminum, and iron, and the non-metallic material is any one of polypropylene (PP), polyethylene (PE), polyvinyl chloride (PVC), fiberglass and ceramics.

Further, a waist compression strap is disposed on the middle vertical support strap, back sides of both ends of the waist compression strap are provided with third hook-and-loop fastener hook patches, and the third hook-and-loop fastener hook patches are detachably fixed on the hook-and-loop fastener loop patch of the waistband.

Further, the limiting assembly is fixedly installed at an upper end of the middle vertical support strap.

Further, a buffer member is embedded in either of the shoulder straps.

Further, the fixed portion has a sleeve structure made of a double-layer material, the fixing member is disposed at a top end of the fixed portion, the sliding portion is inserted into a sleeve of the fixed portion in a clearance fit manner, and the fixing member sequentially passes through a front side of the fixed portion, a sliding groove in the sliding portion, and a back side of the fixed portion.

Further, the limiting assembly includes a front limiting strip, a back limiting strip, and first elastic elements, horizontal grooves are formed in middle portions of the front limiting strip and the back limiting strip, a plurality of engaging positions distributed in an equidistant array are arranged in the horizontal grooves, support columns are disposed on both the front limiting strip and the back limiting strip, the support columns are installed in the horizontal grooves in a slidably fitted manner, the first elastic elements are mounted on the support columns, and the first elastic element on the support column is snap-fitted into the corresponding engaging position of the horizontal groove.

Further, a structure of the first elastic element is any one of an external two-segment type, an external one-segment pressure-opening type and an embedded elastic bead type; and when the support column slides in the horizontal groove, the support column is snap-fitted on the engaging position for limiting after the first elastic element bounces open.

Further, the limiting assembly includes a limiting tube, a limiting column, a fixing clip, and a second elastic element, the limiting column is installed in the limiting tube in a clearance fit manner, a limiting structure is disposed on the limiting column, the fixing clip is mounted on an inner side of the limiting tube, and the second elastic element drives the fixing clip to be cooperatively installed at the limiting structure.

Further, the limiting structure is provided with a plurality of equidistantly distributed holes, the holes are formed on the limiting column, the second elastic element drives the fixing clip to be inserted into the hole in a clearance fit manner, and when the fixing clip is pressed, the second elastic element is in a compressed state, and the fixing clip disengages from the hole such that the limiting column performs telescopic sliding in the limiting tube; and a fourth hook-and-loop fastener hook patch is fixedly mounted on a side of an upper end of the fixed portion close to the sliding portion, the sliding portion is made of a fabric with a hook-and-loop fastener loop patch, and the fourth hook-and-loop fastener hook patch is detachably fixed on the hook-and-loop fastener loop patch of the sliding portion.

Further, the limiting structure employs a serrated groove, an end of the fixing clip away from the second elastic element is provided with a first snap-fit clasp, a middle portion of the first snap-fit clasp is mounted on the limiting tube in a rotatably fitted manner, the first snap-fit clasp is pressed into the serrated groove through the second elastic element, and an outer side surface of the first snap-fit clasp is shaped like an obtuse angle; and when the first snap-fit clasp is pressed, the second elastic element is driven to be compressed between the first snap-fit clasp and the fixing clip, and the first snap-fit clasp disengages from the serrated groove such that the limiting column performs telescopic sliding in the limiting tube.

Further, the fixing clip employs a second snap-fit clasp, the second elastic element employs a third elastic element, the limiting tube is provided with a hollowed-out snap-fit groove, the limiting structure includes an accommodating slot configured to accommodate the second snap-fit clasp and the third elastic element, the second snap-fit clasp is installed in the accommodating slot in a rotatably fitted manner, a plurality of snap-fit groove positions distributed in an equidistant array are arranged in the hollowed-out snap-fit groove, and the third elastic element drives the second snap-fit clasp to be pressed in the snap-fit groove position of the hollowed-out snap-fit groove.

Optionally, the limiting tube and the limiting column are made of a metallic or non-metallic material; and the metallic material is any one of iron, stainless steel, and aluminum, and the non-metallic material is any one of fiberglass, resin, plastic, and ceramics.

Optionally, the limiting tube is any one of a square tube, a cylindrical tube, or an elliptical tube, and the limiting column matches the limiting tube in shape.

Compared with the prior art, the present disclosure has the beneficial effects as follows:

1. Through a cooperative connection between the fixed portion and the sliding portion, in combination with a lifting sliding structure of the fixing member and the sliding groove, the present disclosure achieves continuous adjustability of back support heights, precisely adapts to users of different heights and back lengths, applies to users of different body shapes, fully covers target correction areas, effectively solves the problem of poor adaptability of traditional fixed-length supports, and significantly expands the application scope.

2. Through multiple schemes designed for the limiting assembly (such as the designs of elastic element engagement, perforated insertion, and serrated groove latching), the present disclosure achieves precise positioning after adjustment, prevents displacement or loosening after locking, ensures that the back correction belt always maintains a stable supporting force and correction angle during wearing, and prevents weakening of the correction effect caused by support sliding, thereby enhancing the reliability and durability of correction.

3. The detachable connection between the waistband and the shoulder straps through the hook-and-loop fastener, in combination with the design of the shoulder straps with the embedded buffer members, not only facilitates quick wearing and removal, but also effectively disperses shoulder pressure and prevents local compression injuries; the support strips are embedded in the vertical support straps between the waistband and the fixed portion, which ensures support rigidity, achieves uniform pressure distribution through a plurality of support structures, reduces discomfort in the waist and back, and enhances comfort during long-term wearing; and the adjustable design of the waist compression strap enables to flexibly control a compression force according to user needs, ensuring both the correction effect and wearing experience.

Additional aspects and advantages of the present disclosure will be set forth partially in the following description, which will become obvious in the following description, or may be learned by practice of the present disclosure.

### BRIEF DESCRIPTION OF THE DRAWINGS

In order to more clearly illustrate the technical solutions in the embodiments of the present disclosure or in the prior art, the accompanying drawings required to be used in the embodiments or the prior art description are simply introduced below, and obviously, the accompanying drawings described below are only some embodiments of the present disclosure, such that for those of ordinary skill in the art, other drawings may further be derived from these drawings without making inventive efforts.

FIG. 1 is a schematic structural diagram of a front side of the present disclosure.

FIG. 2 is a schematic structural diagram of a back side of the present disclosure.

FIG. 3 is a schematic structural diagram of a front side of a first limiting assembly.

FIG. 4 is a schematic structural diagram of a back side of FIG. 3.

FIG. 5 is a schematic structural diagram of a two-segment elastic element in FIG. 3.

FIG. 6 is a schematic structural diagram of an external one-segment pressure-opening elastic element in FIG. 3.

FIG. 7 is a schematic structural diagram of an embedded elastic element in FIG. 3.

FIG. 8 is a schematic structural diagram of an embedded elastic bead elastic element in FIG. 3.

FIG. 9 is a schematic structural diagram of a front side of a second limiting assembly.

FIG. 10 is a structural schematic diagram of a fixing clip and a second elastic element in FIG. 9.

FIG. 11 is a schematic structural diagram of a front side of a third limiting assembly.

FIG. 12 is a schematic structural diagram of installation of a first snap-fit clasp and a serrated groove in FIG. 11.

FIG. 13 is a schematic structural diagram of a front side of a fourth limiting assembly.

FIG. 14 is a schematic structural diagram of a back side of FIG. 13.

FIG. 15 is a schematic structural diagram of a front side of a limiting column in FIG. 13.

FIG. 16 is a schematic structural diagram of a back side of a limiting column in FIG. 13.

FIG. 17 is a schematic structural diagram of retracting to a shortest position FIG. 13.

FIG. 18 is a schematic structural diagram of FIG. 13 where a second snap-fit clasp is pulled to an edge of a snap-fit groove position.

FIG. 19 is a schematic structural diagram of FIG. 13 where a second snap-fit clasp is snap-fitted into a snap-fit groove position.

FIG. 20 is a schematic structural diagram of FIG. 19 where a second snap-fit clasp is further pulled down.

FIG. 21 is a schematic structural diagram of when extending to a limit position FIG. 13.

FIG. 22 is a schematic structural diagram of shortening back to an edge of a snap-fit groove position in FIG. 21.

Reference numerals in the figures: 1-waistband; 2-shoulder strap; 3-strap sleeve; 4-fixed portion; 5-sliding portion; 6-fixing member; 7-sliding groove; 8-limiting assembly; 9-first hook-and-loop fastener hook patch; 10-second hook-and-loop fastener hook patch; 11-waist compression strap; 12-third hook-and-loop fastener hook patch; 13-buffer member; 14-front limiting strip; 15-back limiting strip; 16-first elastic element; 17-horizontal groove; 18-engaging position; 19-support column; 20-limiting tube; 21-limiting column; 22-fixing clip; 23-second elastic element; 24-limiting structure; 241-hole; 242-serrated groove; 25-first snap-fit clasp; 26-second snap-fit clasp; 27-third elastic element; 28-hollowed-out snap-fit groove; 29-accommodating slot; 30-snap-fit groove position; 31-vertical support strap; 32-support strip; and 33-fourth hook-and-loop fastener hook patch.

### DETAILED DESCRIPTIONS OF THE EMBODIMENTS

The technical solutions of the present disclosure will be clearly and completely described below in combination with the accompanying drawings. Apparently, the embodiments described are some rather than all of the embodiments of the present disclosure.

The assemblies in the embodiments of the present disclosure described and illustrated in the accompanying drawings usually can be arranged and designed according to various different configurations. Therefore, the following detailed description of the embodiments of the present disclosure provided in the accompanying drawings is not intended to limit the protection scope of the present disclosure, but only to represent the selected embodiments of the present disclosure.

Based on the embodiments of the present disclosure, all the other embodiments obtained by those of ordinary skill in the art without making creative efforts shall fall within the scope of protection of the present disclosure.

In the description of the present disclosure, it should be noted that the terms “center”, “upper”, “lower”, “left”, “right”, “vertical”, “horizontal”, “inner”, “outer”, etc. indicate azimuthal or positional relations based on those shown in the drawings only for ease of description of the present disclosure and for simplicity of description, and are not intended to indicate or imply that the referenced device or element must have a particular orientation and be constructed and operative in a particular orientation, and thus may not be construed as a limitation on the present disclosure. In addition, the terms "first", "second", and "third” are for descriptive purposes only and should not be construed as indicating or implying relative importance.

In the description of the present disclosure, it should be noted that, unless otherwise clearly specified, meanings of terms “mounting”, “connected”, “connecting” should be understood in a board sense. For example, the connection may be a fixed connection, a detachable connection, an integral connection; may be a mechanical connection or an electrical connection; may be a direct connection or an indirect connection by using an intermediate medium; or may be intercommunication between two components. For those of ordinarily skilled in the art, specific meanings of the above terms in the present disclosure could be understood according to specific circumstances.

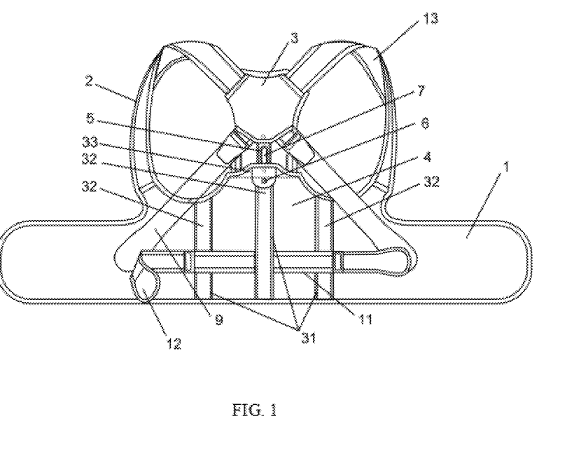

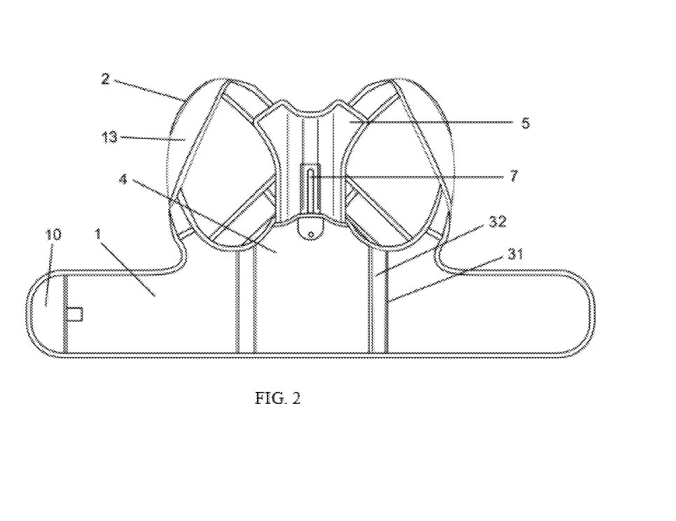

As shown in FIGS. 1 and 2, the present disclosure provides a height-adjustable back correction belt, including a waistband 1, where shoulder straps 2 are disposed on two sides of an upper portion of the waistband 1, and a strap sleeve 3 is mounted between the two shoulder straps 2 in a penetrating manner; and

a fixed portion 4 is disposed in a middle of the upper portion of the waistband 1, a sliding portion 5 is disposed on a rear side of the strap sleeve 3, the sliding portion 5 is located above the fixed portion 4, a fixing member 6 is disposed in the fixed portion 4, a sliding groove 7 is formed in the sliding portion 5, the fixing member 6 is installed in the sliding groove 7 in a liftably and slidably fitted manner, and a limiting assembly 8 is disposed between the fixing member 6 and the sliding groove 7.

An operating principle is as follows: After wearing the waistband 1 and the shoulder straps 2, a user achieves tightness adjustment through sliding fit of the fixing member 6 in the sliding groove 7, thereby enabling adaptability to different groups of users; and arrangement of the limiting assembly 8 ensures that the fixing member 6 is locked in the sliding groove 7 after sliding adjustment, thereby achieving position limiting and better adaptability, effectively covering target correction areas, reducing binding discomfort, and enhancing user experience.

Further, the waistband 1 is made of a fabric with a hook-and-loop fastener loop patch, a back side of either of the shoulder straps 2 is provided with a first hook-and-loop fastener hook patch 9, an end of a back side of the waistband 1 is provided with a second hook-and-loop fastener hook patch 10, and both the first hook-and-loop fastener hook patch 9 and the second hook-and-loop fastener hook patch 10 are detachably fixed on the hook-and-loop fastener loop patch of the waistband 1; and wearing and fixing of the waistband 1 and the shoulder straps 2 may be achieved through an adhesive connection between the hook-and-loop fastener loop patch and the hook-and-loop fastener hook patch, which facilitates use and disassembly of the back correction belt.

Further, a plurality of vertical support straps 31 are arranged between the waistband 1 and the fixed portion 4, and support strips 32 are embedded in the vertical support straps 31, which enhances strength of the fixed portion 4 and prevents easy deformation, thereby improving a correction effect.

Optionally, the support strips 32 are made of a metallic or non-metallic material, the metallic material is any one of stainless steel, aluminum, and iron, and the non-metallic material is any one of polypropylene (PP), polyethylene (PE), polyvinyl chloride (PVC), fiberglass and ceramics; and the support strips 32 may be made of any of the above materials, thereby facilitating actual production and application.

Further, a waist compression strap 11 is disposed on the middle vertical support strap, back sides of both ends of the waist compression strap 11 are provided with third hook-and-loop fastener hook patches 12, and the third hook-and-loop fastener hook patches 12 are detachably fixed on the hook-and-loop fastener loop patch of the waistband 1, which enables to further apply pressure to the waist, thereby improving the correction effect.

Further, the limiting assembly 8 is fixedly installed at an upper end of the middle vertical support strap, which enables to perform lifting adjustment at a middle position, thereby ensuring overall balance and wearing comfort.

Further, a buffer member 13 is embedded in either of the shoulder straps 2, which effectively protects the user’s shoulders and prevents shoulder injury by compression.

Further, the fixed portion 4 has a sleeve structure made of a double-layer material, the fixing member 6 is disposed at a top end of the fixed portion 4, the sliding portion 5 is inserted into a sleeve of the fixed portion 4 in a clearance fit manner, and the fixing member 6 sequentially passes through a front side of the fixed portion 4, the sliding groove 7 in the sliding portion 5, and a back side of the fixed portion 4, which enables to achieve rapid wearing and assembly, and facilitates actual use.

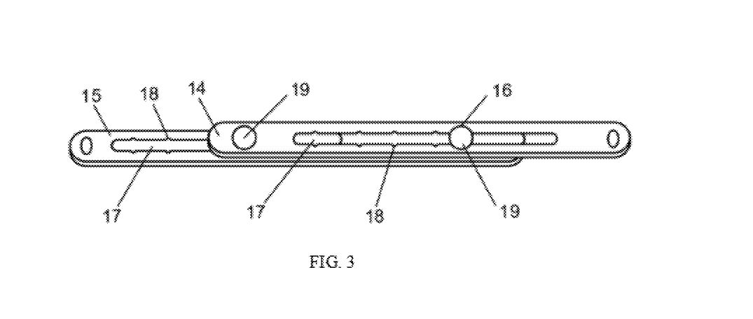

As shown in FIGS. 3 to 8, further, the limiting assembly 8 includes a front limiting strip 14, a back limiting strip 15, and first elastic elements 16, horizontal grooves 17 are formed in middle portions of the front limiting strip 14 and the back limiting strip 15, a plurality of engaging positions 18 distributed in an equidistant array are arranged in the horizontal grooves 17, support columns 19 are disposed on both the front limiting strip 14 and the back limiting strip 15, the support columns 19 are installed in the horizontal grooves 17 in a slidably fitted manner, the first elastic elements 16 are mounted on the support columns 19, and the first elastic element 16 on the support column 19 is snap-fitted into the corresponding engaging position 18 of the horizontal groove 17; and after adjusting and determining a height, the first elastic element 16 on the support column 19 may be snap-fitted into the engaging position 18 corresponding to the height, thereby achieving an effect of locking a height position, and facilitating actual use and operation.

Further, a structure of the first elastic element 16 is any one of an external two-segment type, an external one-segment pressure-opening type and an embedded elastic bead type; when the support column 19 slides in the horizontal groove, the support column 19 is snap-fitted on the engaging position 18 for limiting after the first elastic element 16 bounces open; and any structure of the first elastic element 16 may be selected, thereby achieving high optionality.

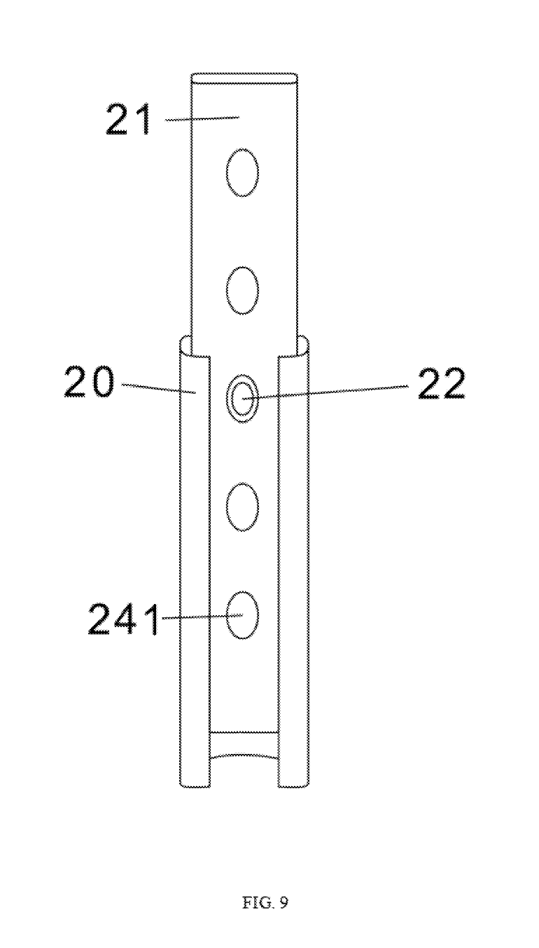

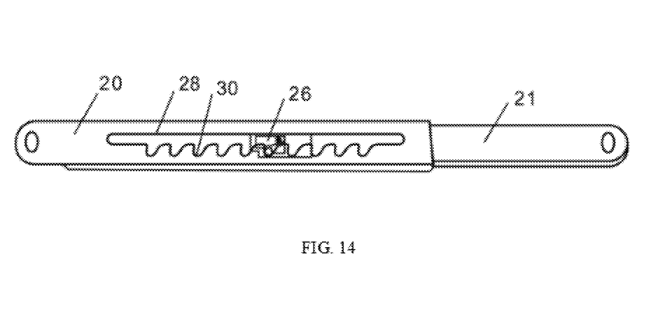

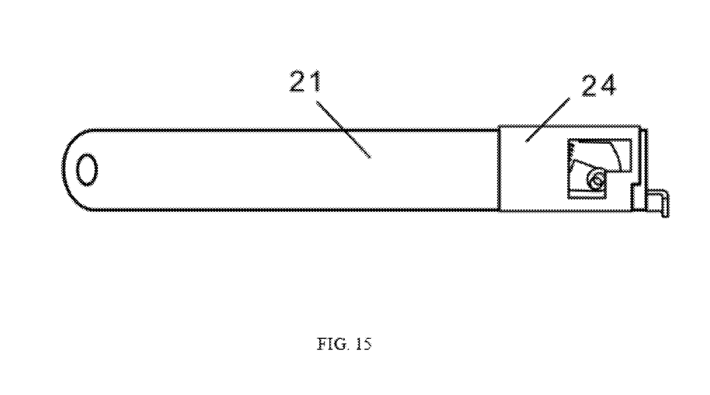

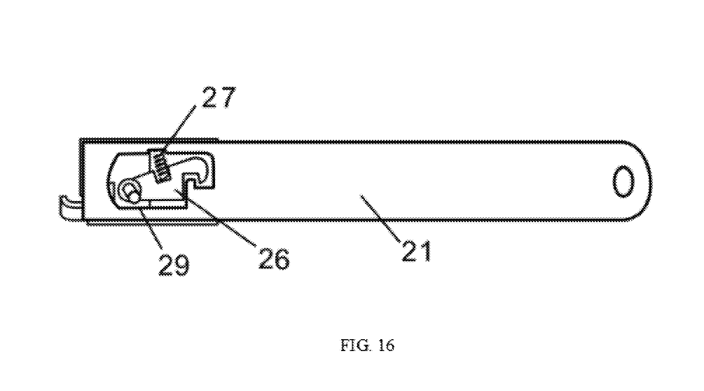

As shown in FIGS. 9 to 16, further, the limiting assembly 8 includes a limiting tube 20, a limiting column 21, a fixing clip 22, and a second elastic element 23, the limiting column 21 is installed in the limiting tube 20 in a clearance fit manner, a limiting structure 24 is disposed on the limiting column 21, the fixing clip 22 is mounted on an inner side of the limiting tube 20, and the second elastic element 23 drives the fixing clip 22 to be cooperatively installed at the limiting structure 24; and height limiting of the limiting column 21 in the limiting tube 20 after telescopic adjustment may be achieved by using a structure of the second limiting assembly 8, which enables to determine a distance between the fixed portion 4 and the sliding portion 5 after wearing.

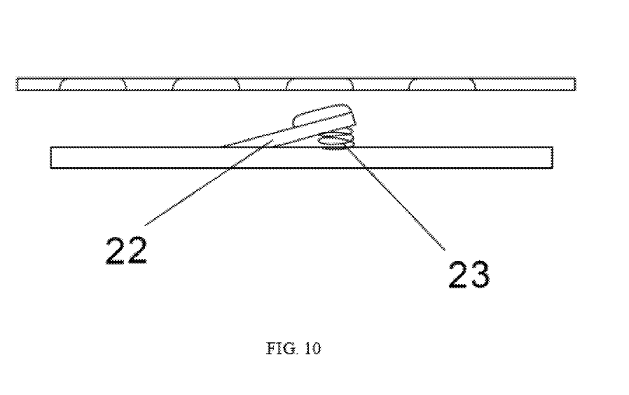

As shown in FIGS. 9 and 10, further, the limiting structure 24 is provided with a plurality of equidistantly distributed holes 241, the holes 241 are formed on the limiting column 21, the second elastic element 23 drives the fixing clip 22 to be inserted into the hole 241 in a clearance fit manner, and when the fixing clip 22 is pressed, the second elastic element 23 is in a compressed state, and the fixing clip 22 disengages from the hole 241 such that the limiting column 21 performs telescopic sliding in the limiting tube 20; height position locking may be achieved by mutual cooperation between the hole 241 and the fixing clip 22; and

as shown in FIG. 1, a fourth hook-and-loop fastener hook patch 33 is fixedly mounted on a side of an upper end of the fixed portion 4 close to the sliding portion 5, the sliding portion 5 is made of a fabric with a hook-and-loop fastener loop patch, and the fourth hook-and-loop fastener hook patch 33 is detachably fixed on the hook-and-loop fastener loop patch of the sliding portion 5; and after a height position is determined, fixing by means of adhesion through the hook-and-loop fastener prevents retraction and displacement of the sliding portion 4, thereby ensuring the correction effect.

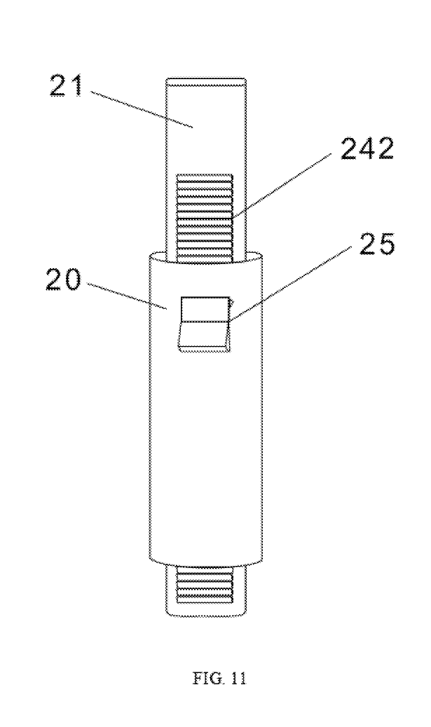

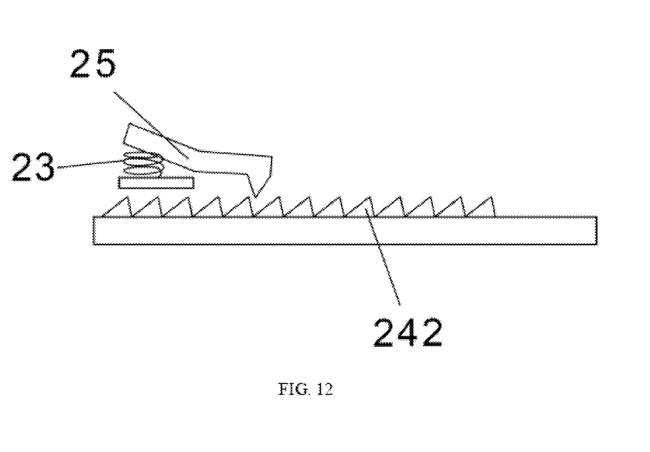

As shown in FIGS. 11 and 12, further, the limiting structure 24 employs a serrated groove 242, an end of the fixing clip 22 away from the second elastic element 23 is provided with a first snap-fit clasp 25, a middle portion of the first snap-fit clasp 25 is mounted on the limiting tube 20 in a rotatably fitted manner, the first snap-fit clasp 25 is pressed into the serrated groove 242 through the second elastic element 23, and an outer side surface of the first snap-fit clasp 25 is shaped like an obtuse angle; when the first snap-fit clasp 25 is pressed, the second elastic element 23 is driven to be compressed between the first snap-fit clasp 25 and the fixing clip 22, and the first snap-fit clasp 25 disengages from the serrated groove 242 such that the limiting column 21 performs telescopic sliding in the limiting tube 20; and mutual cooperation between the serrated groove and the first snap-fit clasp 25 achieves self-locking limitation during insertion, thereby facilitating wearing and use.

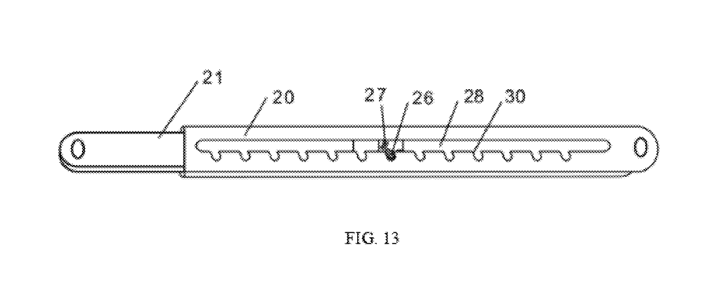

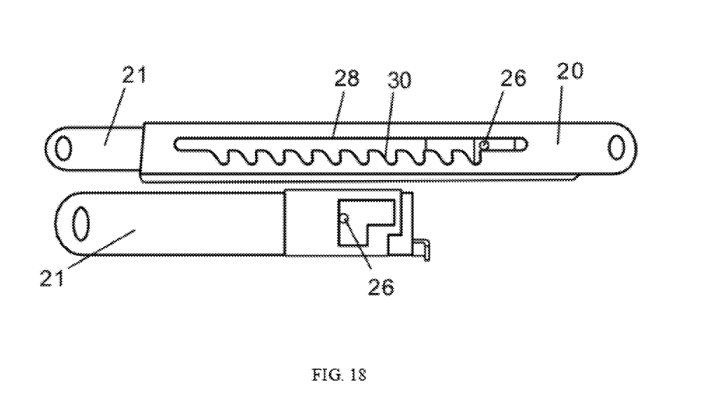

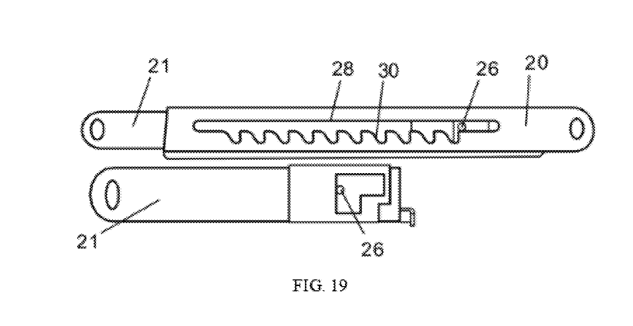

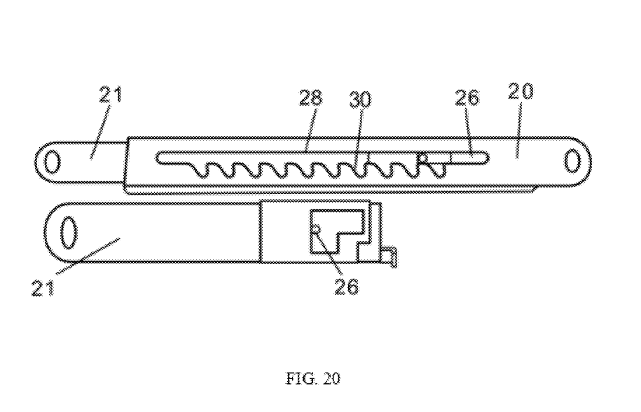

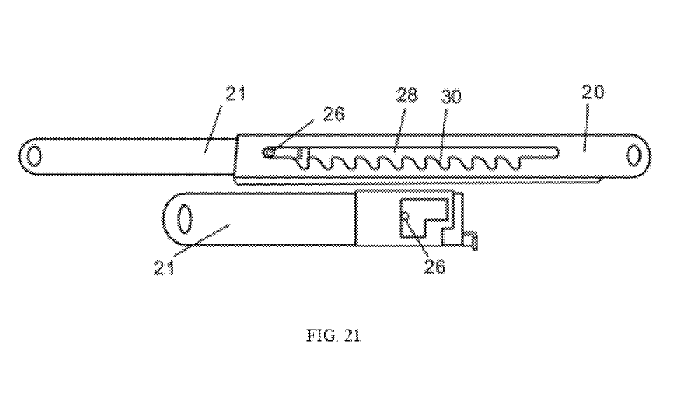

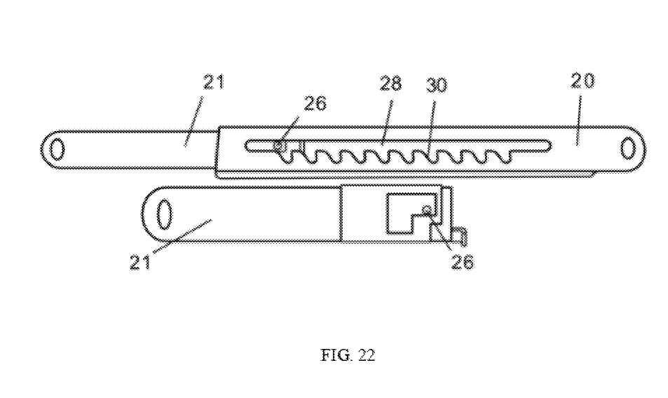

As shown in FIGS. 13 to 22, further, the fixing clip 22 employs a second snap-fit clasp 26, the second elastic element 23 employs a third elastic element 27, the limiting tube 20 is provided with a hollowed-out snap-fit groove 28, the limiting structure 24 includes an accommodating slot 29 configured to accommodate the second snap-fit clasp 26 and the third elastic element 27, the second snap-fit clasp 26 is installed in the accommodating slot 29 in a rotatably fitted manner, a plurality of snap-fit groove positions 30 distributed in an equidistant array are arranged in the hollowed-out snap-fit groove 28, and the third elastic element 27 drives the second snap-fit clasp 26 to be pressed in the snap-fit groove position 30 of the hollowed-out snap-fit groove 28; and a third limiting structure 24 may be formed by related structures of the second snap-fit clasp 26 and the hollowed-out snap-fit groove 28, and the third limiting structure 24, by means of the third elastic element 27, drives the second snap-fit clasp 26 to be snap-fitted into the snap-fit groove position of the hollowed-out snap-fit groove 28 for position limiting, thereby facilitating actual use.

Optionally, the limiting tube 20 and the limiting column 21 are made of a metallic or non-metallic material; the metallic material is any one of iron, stainless steel, and aluminum, and the non-metallic material is any one of fiberglass, resin, plastic, and ceramics; and any of the above materials may be selected for production to ensure the stability of telescopic adjustment and locking.

Optionally, the limiting tube 20 is any one of a square tube, a cylindrical tube, or an elliptical tube, and the limiting column 21 matches the limiting tube 20 in shape; and an appropriate shape and structure may be selected according to actual conditions to achieve stable telescopic adjustment and locking, thereby facilitating the wearing and use of the back correction belt.

An objective of the present disclosure is to provide a height-adjustable back correction belt to solve the problems raised in the background art.

To more clearly express the technical solutions of the present disclosure, the present disclosure discloses the back correction belt, and as shown in FIG. 1, the fixed portion 4 is disposed in a middle of a rear side of the waistband 1, the fixed portion 4 has a sleeve structure composed of two layers of materials, the vertical support straps 31 are arranged on both sides and in a middle of the waistband 1, and the support strips 32 are embedded (metallic materials include stainless steel, aluminum, iron, and the like, and non-metallic materials include PP, PE, PVC, fiberglass, ceramics, and the like), where schemes of the limiting assembly 8 that supports telescopic movement include the following:

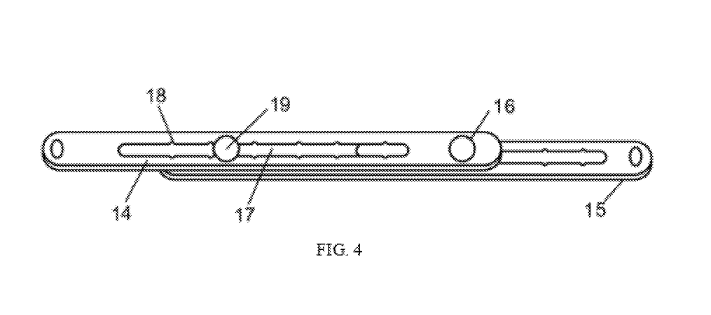

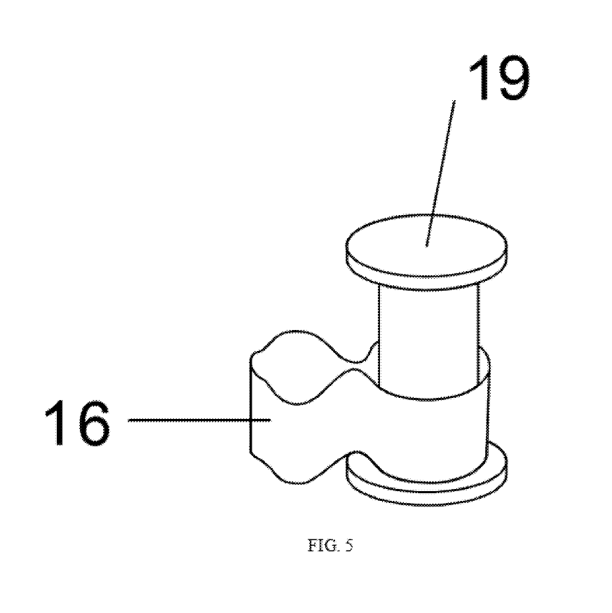

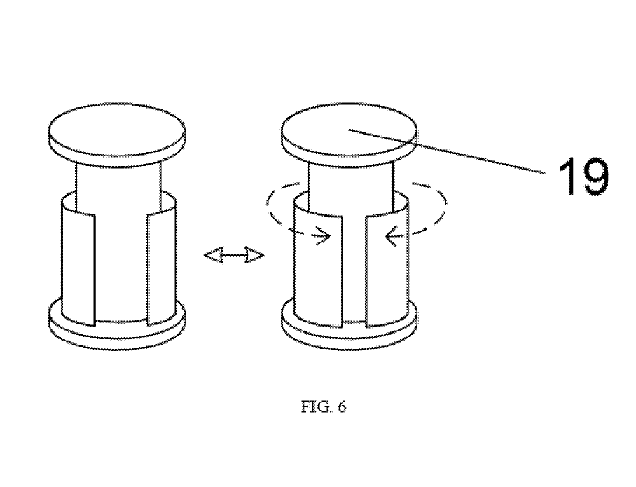

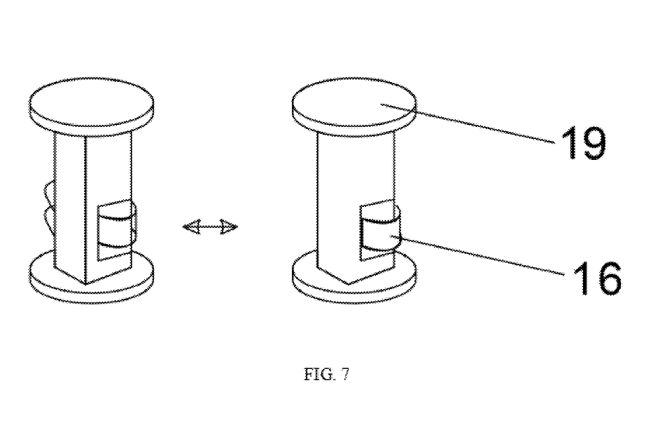

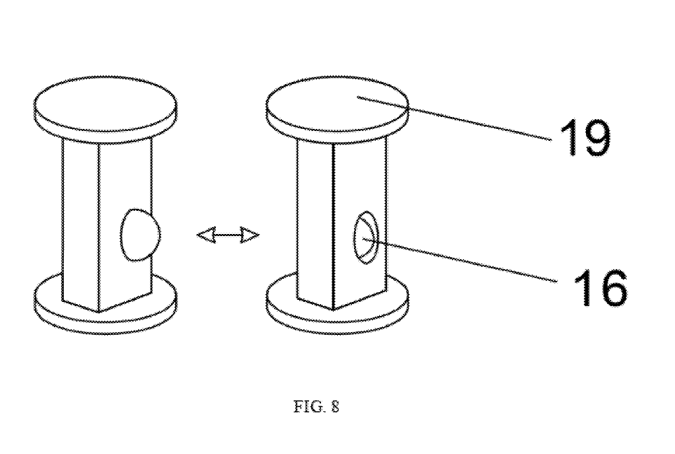

Scheme 1: As shown in FIGS. 3 and 4, the limiting assembly 8 includes a front limiting strip 14 and a back limiting strip 15 (metallic materials include iron, stainless steel, aluminum, and the like, and non-metallic materials include fiberglass, resin, plastic, ceramics, and the like), horizontal grooves 17 are formed in middles of the front limiting strip 14 and the back limiting strip 15, engaging positions 18 are arranged in the horizontal grooves 17 at intervals, support columns 19 are slidably connected in the horizontal grooves 17, and first elastic elements 16 (structurally including an external two-segment elastic element shown in FIG. 5, an external one-segment pressure-opening elastic element shown in FIG. 6, an embedded elastic element shown in FIG. 7, and an embedded elastic bead elastic element shown in FIG. 8) are arranged on the support columns 19; and when sliding in the horizontal groove 17, the support column 19 is snap-fitted on the engaging position 18 after bouncing open through the first elastic element 16, thereby achieving the purpose of limiting. The technical effect of length adjustment is achieved through sliding of the front limiting strip 14 and the back limiting strip 15.

Scheme 2: As shown in FIGS. 9 and 10, the limiting assembly 8 includes a limiting tube 20 and a limiting column 21 (metallic materials include iron, stainless steel, aluminum, and the like, and non-metallic materials include fiberglass, resin, plastic, ceramics, and the like), holes 241 are formed on the limiting column 21 at intervals, a fixing clip 22 is mounted on the limiting tube 20, a second elastic element 23 is mounted on an inner side of the fixing clip 22, and the limiting column 21 moves up and down in the limiting tube 20 after the second elastic element 23 is pressed; and the limiting column 21 is installed in the limiting tube 20 through a hole for position limiting, and the limiting column 21 is pulled after pressing the fixing clip 22 downward to achieve the technical effect of length adjustment.

Scheme 3: As shown in FIGS. 11 and 12, the limiting assembly 8 includes a limiting tube 20 and a limiting column 21 (metallic materials include iron, stainless steel, aluminum, and the like, and non-metallic materials include fiberglass, resin, plastic, ceramics, and the like), a serrated groove 242 is formed in a middle of the limiting column 21, a second snap-fit clasp 26 is mounted on the limiting tube 20, a second elastic element 23 is installed in the second snap-fit clasp 26, and after the second snap-fit clasp 26 is pressed, the second snap-fit clasp 26 disengages from the serrated groove 242, and the limiting column 21 moves up and down in the limiting tube 20; and the limiting column 21 is pulled after pressing the fixing clip 22 downward to achieve the technical effect of length adjustment.

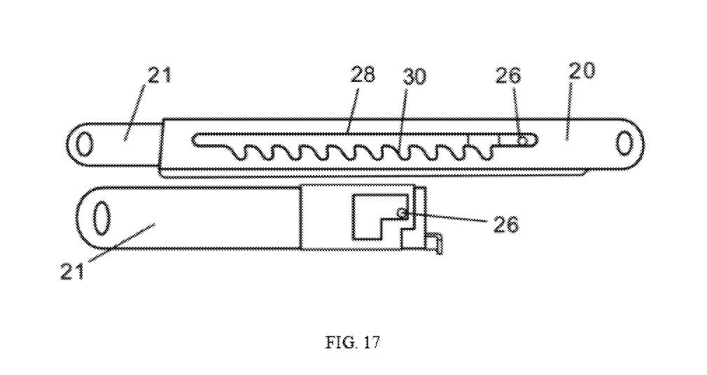

Scheme 4: As shown in FIGS. 13 to 16, the limiting assembly 8 includes a limiting tube 20 and a limiting column 21 (metallic materials include iron, stainless steel, aluminum, and the like, and non-metallic materials include fiberglass, resin, plastic, ceramics, and the like), a second snap-fit clasp 26 and a third elastic element 27 are disposed at a tail portion of an inner side of the limiting column 21, the limiting tube 20 is cylindrical, and hollowed-out snap-fit grooves 28 are correspondingly formed on front and back sides of the limiting tube; the second snap-fit clasp 26 is snap-fitted into the snap-fit groove position 30 of the hollowed-out snap-fit groove 28 through the third elastic element 27 for position limiting, that is, when the limiting column 21 is pulled for telescopic movement, the second snap-fit clasp 26 falls into the snap-fit groove position 30 through the third elastic element 27; and as shown in FIGS. 17 to 22, a movement trajectory of the second snap-fit clasp 26 at different telescopic positions follows the snap-fit groove position 30 of the hollowed-out snap-fit groove 28 for position limiting.

This embodiment does not impose any formal limitation on the shape, material, structure, and any other aspect of the present disclosure. Therefore, any simple alteration, equivalent change and modification which are made to the above embodiments in accordance with the technical essence of the present disclosure all fall within the protection scope of the technical solution of the present disclosure.

What is claimed is:

1. A height-adjustable back correction belt, comprising a waistband, wherein
shoulder straps are disposed on two sides of an upper portion of the waistband, and a strap sleeve is mounted between the two shoulder straps in a penetrating manner; and
a fixed portion is disposed in a middle of an upper portion of the waistband, a sliding portion is disposed on a rear side of the strap sleeve, the sliding portion is located above the fixed portion, a fixing member is disposed in the fixed portion, a sliding groove is formed in the sliding portion, the fixing member is installed in the sliding groove in a liftably and slidably fitted manner, and a limiting assembly is disposed between the fixing member and the sliding groove.

2. The height-adjustable back correction belt according to claim 1, wherein the waistband is made of a fabric with a hook-and-loop fastener loop patch, a back side of either of the shoulder straps is provided with a first hook-and-loop fastener hook patch, an end of a back side of the waistband is provided with a second hook-and-loop fastener hook patch, and both the first hook-and-loop fastener hook patch and the second hook-and-loop fastener hook patch are detachably fixed on the hook-and-loop fastener loop patch of the waistband.

3. The height-adjustable back correction belt according to claim 2, wherein a plurality of vertical support straps are arranged between the waistband and the fixed portion, and support strips are embedded in the vertical support straps.

4. The height-adjustable back correction belt according to claim 3, wherein a waist compression strap is disposed on the middle vertical support strap, back sides of both ends of the waist compression strap are provided with third hook-and-loop fastener hook patches, and the third hook-and-loop fastener hook patches are detachably fixed on the hook-and-loop fastener loop patch of the waistband.

5. The height-adjustable back correction belt according to claim 1, wherein the fixed portion has a sleeve structure made of a double-layer material, the fixing member is disposed at a top end of the fixed portion, the sliding portion is inserted into a sleeve of the fixed portion in a clearance fit manner, and the fixing member sequentially passes through a front side of the fixed portion, a sliding groove in the sliding portion, and a back side of the fixed portion.

6. The height-adjustable back correction belt according to claim 1, wherein the limiting assembly comprises a front limiting strip, a back limiting strip, and first elastic elements, horizontal grooves are formed in middle portions of the front limiting strip and the back limiting strip, a plurality of engaging positions distributed in an equidistant array are arranged in the horizontal grooves, support columns are disposed on both the front limiting strip and the back limiting strip, the support columns are installed in the horizontal grooves in a slidably fitted manner, the first elastic elements are mounted on the support columns, and the first elastic element on the support column is snap-fitted into the corresponding engaging position of the horizontal groove.

7. The height-adjustable back correction belt according to claim 1, wherein the limiting assembly comprises a limiting tube, a limiting column, a fixing clip, and a second elastic element, the limiting column is installed in the limiting tube in a clearance fit manner, a limiting structure is disposed on the limiting column, the fixing clip is mounted on an inner side of the limiting tube, and the second elastic element drives the fixing clip to be cooperatively installed at the limiting structure.

8. The height-adjustable back correction belt according to claim 7, wherein the limiting structure is provided with a plurality of equidistantly distributed holes, the holes are formed on the limiting column, the second elastic element drives the fixing clip to be inserted into the hole in a clearance fit manner, and when the fixing clip is pressed, the second elastic element is in a compressed state, and the fixing clip disengages from the hole such that the limiting column performs telescopic sliding in the limiting tube; and
a fourth hook-and-loop fastener hook patch is fixedly mounted on a side of an upper end of the fixed portion close to the sliding portion, the sliding portion is made of a fabric with a hook-and-loop fastener loop patch, and the fourth hook-and-loop fastener hook patch is detachably fixed on the hook-and-loop fastener loop patch of the sliding portion.

9. The height-adjustable back correction belt according to claim 7, wherein the limiting structure employs a serrated groove, an end of the fixing clip away from the second elastic element is provided with a first snap-fit clasp, a middle portion of the first snap-fit clasp is mounted on the limiting tube in a rotatably fitted manner, the first snap-fit clasp is pressed into the serrated groove through the second elastic element, and an outer side surface of the first snap-fit clasp is shaped like an obtuse angle; and when the first snap-fit clasp is pressed, the second elastic element is driven to be compressed between the first snap-fit clasp and the fixing clip, and the first snap-fit clasp disengages from the serrated groove such that the limiting column performs telescopic sliding in the limiting tube.

10. The height-adjustable back correction belt according to claim 7, wherein the fixing clip employs a second snap-fit clasp, the second elastic element employs a third elastic element, the limiting tube is provided with a hollowed-out snap-fit groove, the limiting structure comprises an accommodating slot configured to accommodate the second snap-fit clasp and the third elastic element, the second snap-fit clasp is installed in the accommodating slot in a rotatably fitted manner, a plurality of snap-fit groove positions distributed in an equidistant array are arranged in the hollowed-out snap-fit groove, and the third elastic element drives the second snap-fit clasp to be pressed in the snap-fit groove position of the hollowed-out snap-fit groove.
